(19) United States
(12) Patent Application Publication
(10) Pub. No.: US 2003/0129166 A1
(43) Pub. Date: Jul. 10, 2003

(54) Human circulating dendritic cell compositions and methods

(75) Inventors: Yu Suen, Irvine, CA (US); Fang-Yao Hou, Irvine, CA (US); James G. Bender, Rancho Santa Margarita, CA (US)

Correspondence Address: CAMPBELL & FLORES LLP, 4370 LA JOLLA VILLAGE DRIVE, 7TH FLOOR, SAN DIEGO, CA, 92122, US

(73) Assignee: Nexell Therapeutics, Inc.

(21) Appl. No.: 10/238,986

(22) Filed: Sep. 9, 2002

Related U.S. Application Data
(63) Continuation of application No. 09/578,532, filed on May 24, 2000, now abandoned.

(51) Int. Cl. A61K 45/00; A61K 38/19; A61K 38/20

(52) U.S. Cl. 424/93.7; 424/85.1; 424/85.2

(57) ABSTRACT

In accordance with the present invention, provided is a method for producing human circulating dendritic cells (cirDC) for therapeutic use, by depleting a human blood leukocyte composition of B cells, T cells and monocytes. Also provided are compositions containing cirDC for therapeutic use.

### BACKGROUND OF THE INVENTION

The present invention relates generally to hematopoietic cells and, more specifically, to methods for producing human circulating dendritic cells for therapeutic use.

Dendritic cells (DCs) are white blood cells that are specialized to present both self and foreign molecules (antigens) to the immune system. Uptake, processing and presentation of antigens by dendritic cells can activate T lymphocytes to recognize and mount an effective immunological attack against cells expressing the antigen.

Although no single surface marker is uniquely associated with dendritic cells, DCs can be distinguished from other hematopoietic cells by their lack of expression of surface marker profiles associated with B cells, T cells, monocytes, NK cells, in combination with high expression of the major histocompatibility antigens. Dendritic cells can also be distinguished from other hematopoietic cells by their ability to stimulate a mixed lymphocyte reaction in vitro with an efficacy of about 100 times that of other hematopoietic cell types.

The ability of dendritic cells to modulate the immune response allows DCs to be used therapeutically in the treatment of infectious diseases and cancer. In one current form of immunotherapy, DCs are pulsed ex vivo with an antigen associated with the infectious agent or tumor cell, to create antigen-pulsed dendritic cells. The antigen-pulsed DCs can be reintroduced into the body to stimulate T lymphocytes in vivo to recognize and attack the pathogenic cells. Antigen-pulsed dendritic cells can also be used to prime large numbers of T lymphocytes ex vivo in a co-culture, and the antigen-specific activated T cells can be introduced into the patient to combat the disease.

Human dendritic cells for use in immunotherapeutic procedures have been produced by culturing peripheral blood mononuclear cells (PBMCs) ex vivo in the presence of hematopoietic growth factors or other additives in order to promote the proliferation and differentiation of dendritic precursor cells into dendritic cells (see, for example, WO 98/06823 and WO 98/06826). Such procedures, while producing clinically relevant numbers of dendritic cells, are laborious and time-consuming, as ex vivo maturation of the dendritic cells requires culturing the cells for many days. It is also unclear whether the DCs obtained by culturing procedures have the identical functional, morphological and phenotypic characteristics as dendritic cells matured in vivo.

Dendritic cells are present in small numbers in a variety of tissues, including lymphoid organs, skin, and circulating blood. Although blood is the most convenient source of dendritic cells, DCs make up only about 1% of the leukocytes in the blood, which has made it difficult to obtain sufficient numbers of high quality blood dendritic cells (cirDC) for therapeutic purposes.

Several methods of enriching for cirDC for research applications have been described. For example, Robinson et al., Eur. J. Immunol. 29:2769-2778 (1999)), describes subjecting buffy coats to serial density gradient centrifugation through stepwise FICOLL or PERCOLL gradients, followed by immunomagnetic depletion of B cells, T cells, monocyte and NK cell populations using CD3, CD14, CD20 and CD16 antibodies. Kohrgruber et al., J. Immunol. 163:3250-3259 (1999), describes FICOLL separation of an apheresis product followed by counterflow elutriation to remove debris and small lymphocytes. The pooled elutriation fractions were immunomagnetically depleted of T, B, NK, hematopoietic stem cells and monocytes using a cocktail of anti-CD3, CD11b, CD16, CD19, CD34 and CD56 antibodies.

Miltenyi Biotec (Gladbach, Germany) sells a blood dendritic cell isolation kit suitable for producing cirDC for research applications. The method involves magnetic depletion of T cell, monocytes and NK cells by retention on a depletion column, followed by positive selection of CD4+ blood dendritic cells using CD4 microbeads. The final positive selection step with CD4 antibody decreases IFN-α production and may cause apoptosis or anergy of the cells (Izaguire et al., Abstract 106, presented at 6^{th} International Workshop on Langerhans Cells, New York (1999)).

Cell separation procedures involving multiple density gradient centrifugation steps can be labor intensive, time consuming, poorly effective and poorly reproducible. Density gradient procedures also can lead to functional alterations of the DCs due to physical trauma during manipulation, or due to extended exposure to the gradient solutions themselves. Furthermore, density gradient procedures used to produce cirDC for therapeutic purposes can be difficult to automate, and also difficult to perform in a closed fluid path system. Preparation of cirDC in a closed fluid path system is optimal for clinical applications, in that the cells are not exposed to environmental contaminants, and the operator is not exposed to any infectious agents present in the cell composition.

Procedures for dendritic cell isolation that require positive selection steps are also disadvantageous, in that the antibody or binding agent used to select or sort the DCs may activate the cells, or otherwise alter the functional properties of the cells. Additionally, incomplete removal of the binding agent may cause adverse immunological reactions upon administration of the cells to humans.

Furthermore, positive selection methods result in isolation of only those DC that express the particular cell surface marker used in the selection procedure. However, it is now understood that there exist at least two distinct populations of cirDC in the blood, which differ quantitatively and qualitatively in expression of cell surface markers. Therefore, cirDC obtained by current positive selection methods may not be fully representative of the cirDC population in vivo.

The procedures currently used to produce cirDC by negative selection require a cocktail of antibodies, usually including antibodies reactive with T cells, B cells, monocytes, NK cells, and often progenitor cells. An effective procedure to produce cirDC in sufficient yield, purity and quality for therapeutic purposes using fewer antibodies has not been described. A simpler procedure would be advantageous in conserving reagents, time and labor.

Thus, there exists a need for a rapid, simple and reproducible method for producing high quality dendritic cells from the blood for use in therapeutic applications. Preferably, the method would avoid density gradient purification and positive selection steps. Ideally, the entire method could be performed in a fully automated, closed fluid path system. The present invention satisfies this need and provides related advantages as well.

### SUMMARY OF THE INVENTION

In accordance with the present invention, provided is a method for producing human circulating dendritic cells (cirDC) for therapeutic use, by depleting a human blood leukocyte composition of B cells, T cells and monocytes. The method is advantageous in that it is amenable to practice in a closed fluid path system, and the cirDC so produced are of sufficient number and quality for use in a variety of therapeutic applications.

Also provided are compositions containing cirDC for therapeutic use. The compositions can advantageously be administered to a patient to induce or enhance beneficial immune responses or to suppress pathogenic immune responses.

### DETAILED DESCRIPTION OF THE INVENTION

The invention provides a method for producing human circulating dendritic cells (cirDC) for therapeutic use, comprising depleting a blood leukocyte composition of B cells, T cells and monocytes. The method is advantageous in that it can be used to simply and rapidly produce large numbers of high quality cirDC that are representative of the dendritic cells in the blood. The method is also advantageous in that density gradient centrifugation and positive selection steps can be avoided, which could alter the functional properties of the cirDC or cause adverse effects upon administration to humans. Furthermore, the method can be fully automated and performed in a closed fluid path system, such that the operator is not exposed to infectious agents present in cell composition, and the cells are not exposed to environmental contaminants.

As used herein, the term “circulating dendritic cell” or “cirDC” refers to a leukocyte obtained from the blood that is characterized phenotypically as CD14− and HLA-DR+. A cirDC can be further characterized as lineage negative (lin−), which indicates that the cirDC does not express surface antigens considered in the art to be characteristic of T cells, B cells, monocytes, NK cells, and hematopoietic progenitor cells. Thus, a cirDC which is lin− can be characterized, for example, as CD3−, CD19−, CD14−, CD16− and CD34−. The surface antigen designations used throughout this disclosure are consistent with the terminology set forth in the Protein Reviews on the Web (PROW) database available on the World Wide Web.

Throughout this disclosure, when referring to cell surface markers (e.g., CD14 antigen and the like), the term “+” is intended to indicate that as assessed by standard phenotyping procedures used in the immunological arts, such as FACS analysis, immunofluorescence or immunohistochemistry, the cells express the recited marker at levels similar to positive control cells. The term “−” indicates that under the same conditions, the cells express the recited marker at levels similar to negative control cells. Exemplary methods to determine whether cells are “+” or “−” for CD3, CD20, CD14, CD11c or HLA-DR are shown in Example 1, below. Antibodies to blood cell surface markers recited herein, which are suitable for phenotyping, are commercially available.

It is now thought that there are two phenotypically and functionally distinct dendritic cell populations in the blood, characterized by the differential expression of the β2 integrin CD11c. These two subsets may reflect different stages of maturation of dendritic cells in the blood, or may alternatively reflect different cell lineages.

CD11c+ cirDC and CD11c− cirDC have been reported to exhibit certain functional, phenotypic and morphological differences. For example, CD11c+ cirDC can be more potent stimulators of allogeneic T cell proliferation than CD11c− cirDC, and can endocytose particulate or soluble antigens more efficiently than CD11c− cells (see, for example, Robinson et al., Eur. J. Immunol. 29:2769-2778 (1999); Kohrgruber et al., J. Immunol. 163:3250-3259 (1999); Pulendran et al., Blood 94:213a (1999)). Furthermore, CD11c+ DCs can preferentially elicit Th1 cytokines, whereas CD11c− DCs can preferentially elicit Th2 cytokines (Pulendran et al., supra (1999)).

Phenotypically, CD11c+ cirDC can be characterized by the expression of certain myeloid markers, such as CD13, CD33, CD32, CLA, or CD11b, which are not expressed, or only expressed at low levels, by CD11c− cirDC. Furthermore, HLA-DR, CD40, CD80 or CD86 can be expressed at higher levels by CD11c+ cirDC than by CD11c− cirDC. Both CD11c+ and Cd11c− cirDC can express CD123 (the interleukin 3 receptor) and CD62L (the ligand for L-selectin) and CD4, although CD11c− can express these molecules at higher levels. The CD11c− cirDC population can also express CD45RA at much higher levels than the CD11c+ cirDC population (see, for example, Robinson et al., supra (1999); Pulendran et al., supra (1999))

Morphologically, CD11c+ cirDC can be characterized by exhibiting an irregular outline and hyperlobulated nucleus by light microscopy, and prominent cytoplasmic processes and lack of prominent ER by electron microscopy. In contrast, CD11c− cirDC possess a rounded morphology, with an oval or indented nucleus and a perinuclear pale zone by light microscopy, and fewer cytoplasmic processes and prominent ER by electron microscopy (see, for example, Robinson et al., supra (1999). Additional morphological features of these two cell types are described in Kohrgruber et al., supra (1999).

The methods of the invention produce human circulating dendritic cells for therapeutic use. As used herein, the phrase “for therapeutic use” refers to cirDC that are in a form and in an amount suitable for administration to humans. Thus, cirDC for therapeutic use are free from contact with substances that could potentially cause adverse immunological reactions in humans administered the cirDC. cirDC for therapeutic use also have not been exposed ex vivo to substances or manipulations that could potentially decrease their efficacy for a desired therapeutic purpose.

In one embodiment, cirDC for therapeutic use are free from contact with binding agents, such as antibodies, that can be present on cirDC obtained by enrichment methods known in the art that involve positive selection or sorting steps. CirDC produced by positive selection methods are generally contacted with binding agents, and either captured on a solid support such as a bead or column and then released from the solid support, or segregated from unwanted cells by a procedure such as fluorescence activated cell sorting (FACS). The binding agent itself, or the method of removing the binding agent from the cell, can alter the function or decrease the viability of the cirDC. If the binding agent is not effectively removed, the residual agent can potentially cause an adverse immunological reaction upon administration to a human. CirDC for therapeutic use that are free from contact with binding agents do not suffer from these disadvantages.

In another embodiment, cirDC for therapeutic use are free from contact with culture reagents, such as serum, non-human animal proteins, growth factors or other additives that can be present on dendritic cells that have been cultured ex vivo, even after washing the cells. CirDC that are free from contact with culture reagents are advantageous in that they have not been exposed to infectious agents, such as prions or viruses, that are potentially present in serum, especially human serum pooled from multiple donors. Additionally, such cirDC are advantageous in that they have not been contacted with animal proteins or other substances that can stimulate the cirDC or cause adverse immunological reactions upon administration to humans. Furthermore, cirDC that are free from contact with culture reagents can be different from cultured cirDC in that they have not proliferated or differentiated ex vivo, which can potentially alter their functional properties compared with the properties of cirDC as they exist in blood.

In a further embodiment, cirDC for therapeutic use are produced in a closed fluid path system. In such a system, the cirDC are not exposed to potential environmental contaminants, such as viruses or microorganisms, or to potential adverse environmental conditions, such as changes in ambient gases, that occur in methods that involve frequent opening of cell containers. As used herein, the term “closed fluid path system” refers to an assembly of components which are closed to the environment. Preferably, a closed fluid path system will include several cell containers, each of each of which is provided with one or more sterile connect-ports for effecting asceptic transfer of cells between the containers, and into and out of the containers, via sterile connect tubing.

An exemplary closed fluid path system for producing cirDC for therapeutic use is the ISOLEX 300i Magnetic Cell Selection System (Nexell Therapeutics Inc., Irvine, Calif.), which can be used to deplete blood leukocytes of B cells, T cells and monocytes, as described further below.

Preferably, all steps from obtaining blood from an individual, to infusing the therapeutic composition into the individual, are carried out in a closed fluid path system. For example, peripheral blood from an individual can be collected and separated using an automated blood cell separator such as the CS3000 cell separator (Fenwal Division, Baxter Healthcare, Deerfield, Ill.), which can be asceptically connected to an ISOLEX 300i. If desired, samples of cells at any stage can be aseptically drawn off from the container system through sterile-connect ports for analysis. In methods involving antigen pulsing of the cirDCs, antigens can be asceptically added to the closed fluid path system through sterile-connect ports. In methods involving co-culturing of antigen pulsed cirDC with T cells, a closed culture container, such as the PL2417 culture bag (Baxter Immunotherapy, Round Lake, Ill.) described in PCT US95/13943, can be asceptically connected to the closed fluid path system. Finally, concentration of the cirDC or antigen-specific T cells into an infusible medium such as PLASMA-LYTE A (Baxter IV Systems, Round Lake, Ill.) can be carried out in the closed fluid path system, and the concentrated cells can be infused directly via the patient's intravenous line without exposing the cells to the environment.

The methods of the invention involve first obtaining a blood leukocyte composition from a human. As used herein, the term “blood leukocyte composition” refers to a composition containing cells obtained from blood, such as from peripheral blood or umbilical cord blood, that is substantially enriched for leukocytes (white blood cells) as compared with whole blood. The cellular composition of normal adult human blood is about 0.1% leukocytes, about 5% platelets, and about 95% red blood cells. Preferably, a blood leukocyte composition is substantially free of red blood cells. More preferably, a blood leukocyte composition is also substantially free of platelets. Thus, in one embodiment, a blood leukocyte composition used in the methods of the invention is a cellular composition of which at least about 70% of the cells, such as at least about 80%, 85%, 90%, 95%, 98% or more, are leukocytes.

Blood leukocytes are composed of mononuclear cells (including lymphocytes, monocytes, stem and progenitor cells, and cirDC) and granulocytes (including neutrophils, eosinophils and basophils). Granulocytes normally comprise about 60-70% of blood leukocytes. Preferably, a starting blood leukocyte composition is substantially free of granulocytes cells. Thus, in one embodiment, a blood leukocyte composition used in the methods of the invention is a cellular composition of which at least about 70% of the cells, such as at least about 80%, 85%, 90%, 95%, 98% or more, are mononuclear cells.

Preferably, to obtain large numbers of blood leukocytes substantially free of granulocytes, the blood leukocyte composition will be a leukapheresis product. Leukapheresis avoids the potential damage and contamination of cells by density gradient procedures such as Ficoll separation. In a typical leukapheresis procedure, using commercially available blood cell separators and manufacturer's recommended settings for mononuclear cell collection, at least 1×10⁹, such as at least 5×10⁹, or 1×10¹⁰ mononuclear cells (MNCs) can be obtained from an individual over the course of several hours. Cell separators suitable for leukapheresis procedures are well known in the art, and include, for example, the Fenwal CS 3000 cell separator (Baxter International Inc, Deerfield, Ill.), the Haemonetics MCS system (Haemonetics Corp., Braintree, Mass.), or the COBE Spectra Apheresis System (Gambro BCT).

Blood from which a blood leukocyte composition is prepared can be obtained from the intended recipient of the ultimate therapeutic composition. Alternatively, blood can be obtained from an HLA-matched donor. For certain therapeutic uses, blood can be obtained from an allogeneic donor. The term “HLA-matched” refers to an individual who expresses some or all of the seven different major histocompatibility complex (MHC) proteins on the cell surface in common with the intended recipient. In contrast, the term “allogeneic” indicates that the donor expresses none or few MHC proteins in common with the intended recipient. Whether or not two individuals are HLA-matched can be determined by standard tissue typing techniques using antibodies or by mixed lymphocyte reactions (MLR).

Procedures that increase the total number of leukocytes in the blood, or that selectively increase the number of cirDC among blood leukocytes, can advantageously be used to increase the number of cirDC for therapeutic use obtained by the methods of the invention. Methods of increasing the number of leukocytes in the blood include, for example, administration of agents that induce the proliferation, differentiation and/or mobilization from the bone marrow of hematopoietic stem or progenitor cells. Agents that increase the number of leukocytes in the blood, by any of these mechanisms, are termed herein “mobilizing agents.” Mobilizing agents include chemotherapeutic agents, irradiation and cytokines, or any combination of these agents. Mobilizing agents can increase the number of leukocytes in the blood by at least 2-fold, such as at least 5-fold, including at least 10-fold as compared with normal blood.

Agents that further increase the number of cirDC represented among blood leukocytes are termed herein “cirDC mobilizing agents.” CirDC mobilizing agents can increase the number of cirDC among blood leukocytes by at least 2-fold, such as at least 5-fold, 10-fold, 20-fold, 30-fold or more as compared with normal blood leukocytes.

Mobilizing agents useful in increasing the number of leukocytes in the blood include the following, alone or in any combination: ligand for the Flt3 receptor (FLT3L), granulocyte colony stimulating factor (G-CSF), granulocyte macrophage colony stimulating factor (GM-CSF), stem-cell factor (SCF), macrophage colony stimulating factor (M-CSF), interleukins (IL-1, IL-2, IL-3, IL-4, IL-5, IL-6, IL-7, IL-8, IL-9, IL-10, IL-11, IL-12, IL-13, IL-14, IL-15), leukemia inhibitory factor (LIF), fibroblast growth factor (FGF), platelet-derived growth factor (PDGF), epidermal growth factor (EGF), transforming growth factor beta (TGFβ), tumor necrosis factor (TNF) interferons (IFN-α, IFNβ and IFN-γ), and agonists of the receptors for any of these molecules, such as daniplestim, progenipoietin (ProGP) and myelopoietin (MPO).

Preferred mobilizing agents for use in the methods of the invention are cirDC mobilizing agents. CirDC mobilizing agents include, for example, FLT3L, G-CSF, GM-CSF, and agonists of the receptors for these cytokines, such as progenipoietin (ProGP) which is a dual receptor agonist of both the G-CSF and the flt3 receptors (Fleming et al., Blood 94:49a (1999)). A particularly preferred mobilizing agent is FLT3L, which is the subject of U.S. Pat. Nos. 5,554,512 and 5,843,423. In preclinical studies, administration of FLT3L was shown to be safe and well tolerated at doses up to 100 μg/kg/day for 14 days, and to increase cirDC levels by up to 30-fold (Lebsack et al., Blood 90:170a (1997)).

Administration of 10 μg/kg/day of FLT3L for 10 consecutive days has been shown to increase cirDC in the blood that are phenotypically CD11c+IL3R− by 48-fold, and cirDC that are phenotypically CD11c−IL3R+ by 13-fold (see Pulendran et al., supra (1999)). Another preferred mobilizing agent is G-CSF. Administration of 10 μg/kg/day of G-CSF for 5 consecutive days has been shown to increase cirDC that are phenotypically CD11c−IL3R+ by 7-fold (see Pulendran et al., supra (1999)).

Mobilizing agents described herein can be obtained in recombinant form from commercial sources and are of sufficient purity for human administration. Alternatively, mobilizing agents can be prepared recombinantly by methods known in the art, given that their nucleic acid sequences are available in public databases and, further, plasmids containing the full-length sequences are commercially available. Mobilizing agents that act as agonists of cytokine receptors can be obtained commercially, designed rationally based on the known receptor structure, or obtained by screening compound libraries.

Appropriate dosages, schedules and routes for administration of mobilizing agents and cirDC mobilizing agents to individuals can be determined by a clinician, and will depend on factors such as the bioactivity of the particular agent, and the health and body weight of the individual.

CirDC comprise about 1% of mononuclear cells in blood of a normal individual not treated with a mobilizing agent. Accordingly, from 1×10⁹ MNC obtained in a typically leukapheresis procedure, about 1×10⁷ are cirDC. The methods of the invention can result in the recovery of at least 10%, such as at least 20%, 40%, 60%, 80%, 90% or more of the cirDC present in a leukapheresis product obtained from an untreated individual. Accordingly, the methods of the invention in an untreated individual can be used to produce at least 1×10⁶ cirDC, such as at least 1×10⁶, 2×10⁶, 4×10⁶, 6×10⁶, 8×10⁶, 9×10⁶ or more cirDC.

An apheresis product obtained from an individual administered a mobilizing agent can contain at least 1×10¹⁰ MNC, such as at least 5×10¹⁰ MNC. Accordingly, starting from an apheresis product obtained from an individual administered a mobilizing agent, given that about 1% of the MNCs are cirDC, and given a recovery of at least 10% of the cirDC, the methods of the invention can be used to obtain at least 1×10⁷ cirDC for therapeutic use, such as at least 5×10⁷ cirDC, 1×10⁸ cirDC, or 5×10⁸ cirDC.

The MNC in an apheresis product obtained from an individual administered a cirDC mobilizing agent can contain at least 2%, such as at least 5%, 10%, 20%, 30% or more cirDC. Starting from 5×10¹⁰ MNC, of which 5% are cirDC, with a recovery of 40% of the cirDC, it is apparent that at least 1×10⁹ cirDC can readily be obtained by the methods of the invention. Depending on the starting number of MNC in the apheresis product, the percentage that are cirDC, and the efficiency of recovery of the cirDC, at least 2×10⁹, such as 5×10⁹, preferably 1×10¹⁰ cirDC for therapeutic use can be obtained by the methods disclosed herein.

The methods of the invention are practiced by depleting a blood leukocyte composition of B cells, T cells and monocytes. As used herein, the term “depleting” refers to any procedure that substantially removes the indicated cell type from the blood leukocyte composition without also substantially removing cirDC from the composition.

The term “substantially removes” with respect to depletion of each of the cell types is intended to mean removal of at least 50% or more of the particular cell type, such as at least 75%, 80%, 90%, 95%, or 97%, including at least 99%, 99.5%, 99.9% or more of the particular cell type. Thus, by depleting B cells, T cells and monocytes from a blood leukocyte composition, the remaining cells are substantially enriched for cirDC. As used herein, the term “substantially enriched” is intended to mean that the cell composition obtained by the method contains at least 50%, preferably at least 70%, more preferably at least 80%, 95%, 97%, 99% or more cirDC for therapeutic use.

The functional, morphological and phenotypic characteristics of B cells (also called B lymphocytes), T cells (also called T lymphocytes), monocytes and other hematopoietic cells are well known in the art and are reviewed in standard immunology textbooks, such as Kuby, Immunology 3rd ed., W. H. Freeman, New York (1997). As used herein, the term “T cell” refers to a leukocyte that is CD3+, the term “B cell” refers to a leukocyte that is CD20+, and the term “monocyte” refers to a leukocyte that is CD14+. These cells will also possess the functional and morphological characteristics of the particular cell type.

A preferred method of depleting a particular cell type involves binding the desired cell with a cell selective binding agent so as to form a complex, and removing the bound complex from the composition. However, other methods of depleting B cells, T cells or monocytes are known in the art or can be readily determined. Such methods, include, for example, erythrocyte resetting (preferably using human erythrocytes), which can be used to deplete T cells; cell size or density separations (eg. counterflow elutriation), which can be used to deplete T cells, B cells or monocytes; complement-mediated cell lysis (eq. using CAMPATH antibody), which can be used to deplete T cells or B cells; adherence to plastic, which can be used to deplete monocytes; and combinations of these methods.

In the methods described herein, B cells, T cells and monocytes, and optionally granulocytes, can be depleted individually in any order, or in any combination. Thus, B cells, T cells and monocytes, and optionally granulocytes, can be depleted sequentially or simultaneously.

As used herein, the term “cell selective binding agent” is a molecule that binds with high affinity to a molecule present on the surface of a recited hematopoietic cell, that is not also substantially present on the surface of a cirDC. Cell selective binding agents bind to molecules present at levels on the indicate cell type that are at least 10-fold, such as at least 100-fold, including at least 1000-fold higher than on cirDC. Methods of determining whether a surface molecule is expressed by a given cell, which will guide the choice of binding agent, are well known in the art and include, for example, immunofluorescence, FACS, radioimmunoassay, immunoprecipitation, mRNA expression analysis, and the like.

A cell selective binding agent need not bind exclusively to the indicated cell type so long as it does not also bind cirDC to a substantial extent. Thus, a cell selective binding agent can bind molecules found on both B cells and T cells, or on all three cell types. A cell selective binding agent can also bind molecules found on other blood cells.

Preferred cell selective binding agents do not activate blood leukocytes. Binding agents that activate leukocytes can induce the production of cytokines that can alter the functional properties of cirDC. Furthermore, residual activated leukocytes obtained together with cirDC can cause adverse effects upon administration to an individual (see, for example, Hsu et al., Transplantation 68:545-554 (1999); and Richards et al., Cancer Res. 59:2096-2101 (1999)).

An exemplary list of molecules present on the surface of B cells is: CD19, CD20, CD21, CD22, CD23, CD24, CD37, CD40, CDw75, CD76, the Ig light chains κ and λ, and the Ig heavy chains γ, α, μ, δ., and ε. Thus, a B cell selective binding agent can be a binding agent that binds any of these molecules, such as an antibody specific for any of these molecules. Preferred B cell selective binding agents bind to CD19, CD20, CD21, CD22 or CD37. Particularly preferred B cell selective binding agents bind to CD19 or CD20.

An exemplary list of molecules present on the surface of T cells is: CD2, CD3, CD4, CD5, CD6, CD7, CD8, CD27, CD28, CD32, CD43, and the T cell receptor α, β, γ or δ chains. Thus, a T cell selective binding agent can be a binding agent that binds any of these molecules, such as an antibody specific for any of these molecules. Preferred T cell selective binding agents bind to CD2, CD3, CD4, CD5, CD7, CD8, or the TCR α or β chains. Particularly preferred T cell selective binding agents bind to CD2 or CD3.

An exemplary list of molecules present on the surface of monocytes is: CDw12, CD13, CD14, CD15, CDw17, CD31, CD32, CD33, CD64, CD98. Thus, a monocyte selective binding agent can be a binding agent that binds any of these molecules, such as an antibody specific for any of these molecules. A preferred monocyte selective binding agent binds to CD14.

The blood leukocyte composition can optionally be further depleted of granulocytes using at least one granulocyte selective binding agent. An exemplary list of molecules present on the surface of granulocytes is CD66b, CD15, CD24, and the like. Thus, a granulocyte selective binding agent can be a binding agent that binds any of these molecules, such as an antibody specific for CD66b, CD15, or CD24. Depleting the blood leukocyte composition of granulocytes using a granulocyte selective binding agent is particularly advantageous when the starting blood leukocyte composition contains a significant number of mature or immature granulocytes. For example, when blood is obtained from an individual administered a mobilizing agent such as G-CSF, GM-CSF, or progenipoietin (ProGP), a blood leukocyte composition can contain a large number of mature and immature granulocytes. Immature granulocytes can be difficult to separate from mononuclear cells using cell separators, but can advantageously be depleted using a granulocyte selective binding agent. Those skilled in the art can readily determine the desireability of depleting the blood leukocyte composition of granulocytes using a granulocyte selective binding agent.

A binding agent useful in the methods of the invention will form a high affinity binding complex with the target cell. As used herein, the term “complex” refers to an interaction between the binding agent and the target cell that has a dissociation constant (Kd) of less than about 10⁻⁵ M, such as less than about 10⁻⁷ M, including less than about 10⁻⁹ M. A preferred binding agent is an antibody, such as a monoclonal, recombinant or single chain antibody, or an antigen binding fragment therefrom, which forms a high affinity complexes with target molecules. Antibodies suitable for use in the methods of the invention are commercially available, or can be produced with high affinity for a desired surface molecule by methods known in the art. Such antibodies can be derived from a single species, including human, rodent, sheep and goat, or can be chimeric.

Preferred cell selective binding agents bind to all or to the majority of the indicated cell type. However, combinations of cell selective binding agents can be used to more completely deplete a particular cell type. As an example, CD4 is expressed on about 65% of T cells, with the remainder expressing CD8. Thus, a combination of binding agents that bind CD4 and CD8 can be used to deplete T cells.

Cell selective binding agents other than antibodies can also be used in the methods of the invention. Such binding agents include lectins, such as soybean agglutinin, which binds to T cells and B cells. Commercially available libraries of small molecule or macromolecular compounds can also be screened using whole B cells, T cells or monocytes, or membranes or isolated surface molecules therefrom, to identify other binding agents. Methods of screening and selecting for binding compounds, including automated screening and selection methods, are well known in the art. The particular method employed will depend on the nature of the compounds being screened. Thus, a cell selective binding agent can be essentially any chemical or biological compound with the appropriate selectivity and affinity for the desired cell, such as a nucleic acid, peptide, peptidomimetic, small organic molecule, or the like.

In one embodiment, target cells are contacted with a binding agent under conditions where complexes are formed between the binding agent and the target cell. Such conditions can be determined by the practitioner, and will depend on factors such as the nature and affinity of the binding agent, the volume of the blood leukocyte composition, and the number of target cells and contaminating cells in the composition. As an example, conditions suitable to form a complex between a binding agent and a target cell are conditions equivalent to contacting 1×10⁷ mononuclear cells in a 1 ml volume with 1.5 μg monoclonal antibody for 30 mins. at room temperature.

In one embodiment, an invention depleting method consists of contacting blood leukocytes with binding agents selective for T cells, B cells and monocytes, with no other depleting steps. In an alternative embodiment, an invention depleting method comprises or consists of contacting blood leukocytes with binding agents selective for two cell types selected from the group consisting of T cells, B cells and monocytes. For example, blood leukocytes optionally are not also contacted with a natural killer (NK) cell selective binding agent, such as an antibody specific for CD16, CD56, or for other molecules present in abundance on NK cells that are not present at significant levels on cirDC. As a further example, blood leukocytes optionally are not also contacted with a stem cell selective binding agent, such as an antibody specific for CD34, or for other molecules present in abundance on stem cells that are not present at significant levels on cirDC. Practicing the methods of the invention with the minimum number of reagents and steps possible is advantageous in saving time, money and handling of the cells.

In an alternative embodiment, an invention depleting method consists of contacting blood leukocytes with one or more binding agents selective for T cells, B cells, monocytes and granulocytes, with no other depleting steps. In another alternative embodiment, depleting consists of contacting blood leukocytes with binding agents selective for two cell types selected from the group consisting of T cells, B cells and monocytes, and additionally contacting blood leukocytes with a binding agent selective for granulocytes, with no other depleting steps. Thus, a method for producing human circulating dendritic cells for therapeutic use can comprise, or consist of, contacting blood leukocytes with binding agents selective for T cells, monocytes and granulocytes. As described previously, the use of granulocyte selective binding agents to deplete granulocytes is particularly advantageous when the starting blood leukocyte composition contains a significant number of mature or immature granulocytes, such as when the blood has been obtained from an individual administered a mobilizing agent that increases granulocyte number.

Following contacting the target cell with the cell selective binding agent, the complex of the binding agent and cell is removed, thus depleting the target cell from the composition. A variety of methods are known in the art to remove binding agent-cell complexes from compositions.

For example, the binding agent can be labeled with a detectable moiety, such as a fluorochrome, and the complexes separated by flow cytometry using a sorter that separates cells having the detectable moiety from those that do not, such as a fluroescence activated cell sorter (FACS). Alternatively, removal of the complex can involve linking the binding agent, either directly or through a secondary binding agent, to a solid support that allows the complex to be separated from unbound cells in the suspension by virtue of binding affinity, density, magnetism or other physical property.

As used herein, the term “secondary binding agent” refers to any molecule or combination of molecules that provides a means of linking the binding agent to the solid support. Exemplary secondary binding agents include antibodies, which can be prepared by known methods so as to have affinity for virtually any cell selective binding agent and can be linked directly to solid supports; biotin and avidin, one of which can be linked to a binding agent and the other of which can be linked to a solid support; Protein A or Protein G, which have affinity for antibodies and can be linked to solid supports, and the like.

Exemplary solid supports include paramagnetic beads, which allow the complexes to be removed with a magnet; chromatography columns and hollow fibers, which allow the complexes to be removed by virtue of size, density or affinity to the matrix; and polystyrene surfaces, which allow the complexes to be removed by panning methods. A variety of secondary binding agents and compatible solid supports are commercially available or can be readily prepared for a particular application.

In one embodiment, the solid support is directly attached to the binding agent. For example, a cell selective binding agent can be conjugated to a paramagnetic bead, and the complex removed from the composition with a magnet. In another embodiment, the solid support is attached to a secondary binding agent. For example, a target cell-binding agent complex can be further contacted with a secondary binding agent (eg. an antibody) conjugated to a paramagnetic bead, and the cell-binding agent-secondary binding agent complex removed from the composition with a magnet.

Paramagnetic beads, antibody-bound paramagnetic beads, magnets and automated systems for magnetic cell separation are commercially available, and detailed protocols for their use are available from the suppliers.

In a preferred embodiment, all depletion steps are conducted in a magnetic cell separation apparatus as described, for example, in U.S. Pat. No. 5,536,475. An exemplary apparatus is the ISOLEX 300i fully automated magnetic cell separation system (Nexell Therapeutics, Inc., Irvine Calif.). Suitable binding agents for use in such an apparatus include cell-specific GMP antibodies, which are commercially available. Depletion in a magnetic cell separation apparatus can be performed, for example, using sheep anti-mouse polyclonal antibodies and paramagnetic beads produced by Dynal A/S (Oslo, Norway).

The methods described above produce a cirDC population for therapeutic use that contains both CD11c+ and CD11c− cirDC. If desired for a particular application, either CD11c+ cirDC or CD11c− cirDC can be further enriched using binding agents selective for the cell surface markers preferentially expressed by the unwanted cell population, and similar depletion methods as described.

In one embodiment, the cirDC are further depleted of CD11c+ cirDC to produce CD11c− cirDC for therapeutic use. As an example, CD11c+ cirDC can be depleted by contacting cirDC with CD11c specific antibodies to form a complex, contacting the complex with secondary antibodies linked to paramagnetic beads, and removing the complexes with magnets. In an alternative embodiment, cirDC are further depleted of CD11c− cirDC to produce CD11c+ cirDC for therapeutic use. As an example, CD11c− cirDC can be depleted by contacting cirDC with CD45RA specific antibodies to form a complex, contacting the complex with secondary antibodies linked to paramagnetic beads, and removing the complexes with magnets.

Optionally, the cirDC produced by the methods of the invention are washed with sterile buffers, concentrated and suspended in an infusible medium before use. The cirDC can be infused into the recipient by a variety of routes, such as into the blood, into a lymph node, or by intradermal or subcutaneous administration (see, for example, Morse et al., Cancer Res. 59:56-58 (1999)).

The cirDC can be used in a variety of therapeutic applications, including in therapeutic applications where dendritic cells produced by other methods are useful. For example, the cirDC can first be pulsed with a desired antigen ex vivo, using methods known in the art for pulsing dendritic cells, and used to induce or enhance an immune response against the antigen so as to prevent or ameliorate a pathological condition (see, for example, Morse et al., Clin. Cancer Res. 5:1331-1338 (1999); Nestle et al., Nature Med. 4:328-332 (1998)). In an exemplary method of preparing antigen-pulsed cir DC, the cirDC, at a concentration of several million/ml, can be co-incubated with antigen, at a concentration of about 10-200 μg/ml, for a period of from several hours to several days.

Exemplary antigens for pulsing of cirDC include products of oncogenes, viral proteins, cell lysates, and normal cellular components that are either modified or aberrantly expressed in a pathology. Contemplated antigens for use in cancer therapy include, for example, whole antigens, peptides or mRNA derived from carcinoembryonic antigen (CEA) (e.g. for breast or colon cancer); Her2/neu (e.g. for breast or ovarian cancer); prostate specific antigen (PSA) and prostate specific membrane antigen (PMSA) (e.g. for prostate cancer); MUC (e.g. for breast cancer); MAGE, GP100, tyrosinase or MART1 (e.g. for melanoma); and tumor cell lysates (e.g. for renal or liver cancer) or apoptotic tumor cells.

Contemplated antigens for use in the prevention or treatment of infectious conditions include human immunodeficiency virus (e.g. HIV-1 and HIV-2), hepatitis B virus, hepatitis C virus, papilloma virus, cytomegalovirus, Epstein-Barr virus, and chlamydia, as well as antigenic preparations therefrom.

The antigen-pulsed cirDC produced by the methods of the invention will acquire the exogenous antigen, process it into peptides and, upon infusion into the patient, present the peptides to T cells in the context of MHC molecules to induce an immune response against the tumor or infected cell. For such an application at least about 10⁶, preferably at least about 10⁷, more preferably at least about 10⁸ antigen-pulsed cirDC can be used.

Alternatively, the antigen-pulsed cirDC can be co-cultured for a suitable period of time, such as from several hours to several days, with T lymphocytes, to produce antigen-specific T cells. Such T cells are activated by contact with the antigen-pulsed T cells, and will induce an immune response against cells expressing the target antigen on their surface when infused into an individual. The T cells can be obtained, by methods known in the art, from the same donor whose blood leukocytes yielded the DC, or from an HLA-matched individual as described above. A T cell population for antigen-pulsing can contain both cytotoxic T cells (CD8+ T cells) and helper T cells (CD4+ T cells) or, using preselection methods known in the art, can contain primarily cytotoxic T cells.

When cirDC are intended to be used as stimulators of T cells ex vivo, the number of antigen-pulsed cirDC required can be in the range of about 0.5 million to about 100 million. This range is based on the assumption that a ratio of 1:5 to 1:10 DC:T-cells is required for efficient activation of the T-cells. It is estimated that about 10 million to 1 billion antigen-specific T-cells are required to achieve the desired cell-killing activity in vivo, and that activated T-cells will comprise about 10% of the total T-cells in a co-culture. Therefore, about 100 million to 10 billion T-cells are needed in the final co-culture. Assuming a proliferation index (PI) of 10 for the T-cells in culture (although a PI of from 10-50 is expected), about 5 million to about 1 billion T-cells are seeded in the beginning co-culture. Thus, at a ratio of 1:10, DC:T-cells, from about 0.5 million to about 100 million antigen-pulsed DC are needed in the co-culture.

The cirDC of the invention can also be used therapeutically without antigen-pulsing. For example, cirDC can be administered in applications where enhancement of the immune system is desired, such as to reconstitute the immune system after bone marrow transplantation. Additionally, cirDC can be administered together with, prior to, or following treatment of a tumor with an agent that induces tumor apoptosis (e.g. a chemotherapeutic agent or irradiation). In such an application, the administered cirDC can take up and present tumor antigens from the apoptosed tumor cells so as to activate the immune system to kill residual tumor cells and/or prevent tumor metastases.

Furthermore, cirDC can be used in a variety of immunosuppressive applications, including in the therapy of autoimmune diseases and in promoting tolerance to transplanted tissues (see, for example, Thomson et al., Transplantation 68:1-8 (1999); U.S. Pat. No. 5,871,728). For example, cirDC obtained from an allogeneic tissue donor can be administered to the tissue recipient so as to reduce the likelihood of rejection of the allograft. For tolerogenic applications, it can be advantageous to first treat the cirDC with an agent, such as TGFβ, IL-10 or cyclosporine A, that decreases expression by the cirDC of co-stimulatory molecules and immunostimulatory cytokines.

It is understood that modifications which do not substantially affect the activity of the various embodiments of this invention are also included within the definition of the invention provided herein. Accordingly, the following examples are intended to illustrate but not limit the present invention.

### EXAMPLE I

### Small-scale Preparation of cirDC

This example shows the preparation of cirDC by depleting blood leukocytes of T cells, B cells and monocytes.

Platelet-washed peripheral blood mononuclear cells (PBMCs) (1×10⁷ cells in 1 ml) were resuspended in phosphate buffered saline (PBS; Biowhittaker) containing 1% human serum albumin (HSA) and 12% sodium citrate. Cells were contacted sequentially with mouse CD2 antibody (Nexell Therapeutics), mouse CD19 antibody (Nexeli Therapeutics), or mouse CD14 antibody (Diaclone) for 30 min at room temperature (RT). Each primary antibody was used at a concentration of 1.5 μg/10⁷ cells/ml. After incubation with each primary antibody, the cells were washed with PBS containing 1% HSA and 12% sodium citrate to remove primary antibodies and incubated with sheep anti-mouse paramagnetic beads (SAM beads; Dynal) at a 2 bead:PBMC ratio for 30 min at RT. The bead/cell rosettes were washed and removed using an MPC⁷-1 Dynal Magnetic Particle Concentrator.

The unbound cells were collected, washed, resuspended and analyzed by FACS analysis for expression of surface markers, using the following labeled antibodies, obtained from Becton Dickinson, following the manufacturer's recommended procedures: anti-IgG1 FITC/anti-IgG1 PE (control); anti-CD2 FITC; anti-CD3 FITC; anti-CD14 FITC; anti-CD19-FITC; anti-CD20 FITC; anti-CD11c FITC/anti-DR PE/anti-CD14 PCP. FACS data was acquired using a FACScan™ flow cytometer (Becton Dickinson). The percentage of cells that were CD3+ (ie. T cells), CD20+ (ie. B cells), CD14+ (ie. monocytes), CD16+56+ (i.e. NK cells), or CD11c+/HLA-DR/CD14− (ie. a subpopulation of cirDC) following various depletion steps in a single experiment is shown in Table 1, and the absolute numbers of each cell type are shown in Table 2.

TABLE 1

| | | | | | CirDC |
|---|---|---|---|---|---|
| % | CD3 | CD20 | CD14 | NK | (CD11c+/DR/14+) |
| pre | 63.72 | 10.87 | 11.67 | 7.84 | 2.05 |
| post CD2 | 2.56 | | 53.84 | 9.42 | 5.63 |
| post CD19 | 66.96 | 0.04 | 18.38 | | 2.23 |
| post CD14 | | | 0.21 | | 1.86 |
| post CD2/19/14 | 10.96 | 0.31 | 4.38 | | 16.15 |

TABLE 2

| Cell # | CD3 | CD20 | CD14 | NK | cirDC |
|---|---|---|---|---|---|
| 2.26E+07 pre | 1.44E+07 | 2.46E+06 | 2.64E+06 | 1.78E+06 | 4.64E+05 |
| 2.16E+06 post CD2 | 5.54E+04 | | 1.17E+06 | 2.04E+05 | 1.22E+05 |
| 5.89E+06 post CD19 | 3.94E+06 | 2.36E+03 | 1.08E+06 | | 1.31E+05 |
| 5.07E+06 post CD14 | | | 1.06E+04 | | 9.42E+04 |
| 1.30E+06 post CD2/19/14 | 1.42E+05 | 4.02E+03 | 5.69E+04 | | 2.10E+05 |

These results show that by contacting peripheral blood mononuclear cells from an untreated individual with binding agents selective for B cells, T cells and monocytes, and removing the complexes from the composition, a cell population enriched for CD11c+/HLA-DR+/CD14− cirDC by at least about 8-fold can be produced. These results further show that at least 45% of the CD11c+/HLA-DR+/CD14− cirDC in the starting population can be recovered by these methods.

The results obtained from the above experiment and four additional experiments, using either CD2 or CD3 antibodies to deplete T cells, are shown in Tables 3 and 4, below. The data presented in Table 4 shows an average recovery of about 52% of cirDC by depleting a human blood leukocyte composition of B cells, T cells and monocytes using CD2, CD19 and CD14 antibodies, and an average recovery of about 58% of cirDC using CD3, CD19 and CD14 antibodies.

TABLE 3

| | donor 2 | | | | | |
|---|---|---|---|---|---|---|
| | | | | | | cirDC (CD11c+/ |
| % | CD2 | CD3 | CD20 | CD14 | NK | DR/14+) |
| pre | 74.83 | 59.91 | 8.72 | 9.19 | 18.53 | 1.39 |
| post CD2 | | 2.67 | | | 16.43 | 2.57 |
| post CD3 | 45.03 | | 16.95 | | | 2.59 |
| post CD19 | | 62.07 | 0.06 | | | 1.00 |
| post CD14 | | | | | | 1.14 |
| post CD2/19/14 | | 5.82 | 37.38 | | | 4.39 |
| post CD3/19/14 | 58.86 | | 18.84 | | | 2.80 |

TABLE 4

| | donor 2 | | | | | cirDC (CD11c+ |
|---|---|---|---|---|---|---|
| cell # | CD2 | CD3 | CD20 | CD14 | NK | /DR/14+) |
| 2.0E7pre | 1.50E+07 | 1.20E+07 | 1.74E+06 | 1.84E+06 | 3.71E+06 | 2.78E+05 |
| 2.4E6post CD2 | | 6.41E+04 | | | 3.94E+05 | 6.17E+04 |
| 3 2E6post CD3 | 1.44E+06 | | 5.42E+05 | | | 8.29E+04 |
| 6.9E6post CD19 | | 4.28E+06 | 4.14E+05 | | | 6.90E+04 |
| 6.2E6post CD14 | | | | | | 7.07E+04 |
| 3.3E6post CD2/19/14 | | 1.92E+05 | 1.23E+06 | | | 1.45E+05 |
| 5.8E6post | 3.41E+06 | | 1.09E+06 | | | 1.62E+05 |
| CD3/19/14 | | | | | | |

### EXAMPLE II

### Large-scale Preparation of cirDC

This example shows the preparation of cirDC for therapeutic use.

Apheresis samples from individuals administered 10 μg/kg/ml FLT3L (Immunex) for 10 days are collected using a Fenwal CS-3000 Cell Separator. Peripheral blood mononuclear cells (PBMCs) (1×10¹⁰ cells) are resuspended in PBS containing 1% HSA and 12% sodium citrate. Cells are sensitized sequentially with 1 mg CD2 antibody (Nexell Therapeutics), 1 mg CD19 antibody (Nexell Therapeutics), or 1 mg CD14 antibody (Diaclone) for 30 min at room temperature (RT), or with all three antibodies together, in an ISOLEX 300i cell selection device. Unbound antibodies are removed by washing and the antibody sensitized cells are incubated with sheep anti-mouse paramagnetic beads (Dynal) at a 2 bead:PBMC ratio for 30 min at RT. The bead/cell rosettes are washed and the unbound cells are collected, washed and resuspended.

From a FLT3L-mobilized donor, at least 10%, such as about 60% of the PBMCs are cirDC. Thus, given a recovery of at least 50% of the starting cirDC, at least 5×10⁸ cirDC, such as about 3×10⁹ cirDC for therapeutic use are obtained from a single apheresis product from a FLT3L-mobilized donor containing 10¹⁰ cells. These cirDC effectively process and present antigen, as assessed by pinocytosis assays, activity in allogeneic mixed lymphocyte assays, and antigen-induced T cell proliferation assays.

Throughout this application various patents and publications have been referenced. The disclosures of these patents and publications in their entireties are hereby incorporated by reference in this application in order to more fully describe the state of the art to which this invention pertains.

Although the invention has been described with reference to the disclosed embodiments, those skilled in the art will readily appreciate that the specific experiments detailed are only illustrative of the invention. It should be understood that various modifications can be made without departing from the spirit of the invention. Accordingly, the invention is limited only by the following claims.

What is claimed is:

1. A method for producing human circulating dendritic cells (cirDC) for therapeutic use, comprising depleting a human blood leukocyte composition of B cells, T cells and monocytes.

2. The method of claim 1, wherein said blood leukocyte composition is substantially free of granulobytes.

3. The method of claim 1, wherein said blood leukocyte composition is obtained from an individual administered at least one mobilizing agent.

4. The method of claim 3, wherein said mobilizing agent is selected from the group consisting of FLT3L, G-CSF, GM-CSF, SCF, M-CSF, IL-1, IL-2, IL-3, IL-4, IL-5, IL-6, IL-7, IL-8, IL-9, IL-10, IL-11, IL-12, IL-13, IL-14, IL-15, LIF, FGF, TNF, ProGP, FGF, PDGF, EGF, TGF, interferon, daniplestim, progenipoietin (ProGP) and myelopoietin (MPO).

5. The method of claim 4, wherein said mobilizing agent is a cirDC mobilizing agent.

6. The method of claim 5, wherein said cirDC mobilizing agent is FLT3L.

7. The method of claim 1, wherein said blood leukocyte composition comprises at least 1×10⁹ mononuclear cells.

8. The method of claim 1, wherein said cirDC for therapeutic use are free from contact with binding agents.

9. The method of claim 1, wherein said cirDC for therapeutic use are free from contact with serum and non-human animal proteins.

10. The method of claim 1, wherein said depleting occurs in a closed fluid path system.

11. The method of claim 1, wherein said cirDC for therapeutic use comprise at least 1×10⁶ cirDC.

12. The method of claim 3, wherein said blood leukocyte composition comprises at least 1×10¹⁰ mononuclear cells.

13. The method of claim 12, wherein said cirDC for therapeutic use comprise at least 1×10⁷ cirDC.

14. The method of claim 5, wherein said cirDC for therapeutic use comprise at least 1×10⁸ cirDC.

15. The method of claim 6, wherein said cirDC for therapeutic use comprise at least 1×10⁹ cirDC.

16. The method of claim 1, wherein said depleting comprises:
(a) contacting: said B cells with at least one B cell selective binding agent; said T cells with at least one T cell selective binding agent; and said monocytes with at least one monocyte selective binding agent, under conditions where complexes are formed between said B cells and said B cell selective binding agent, said T cells and said T cell selective binding agent, and said monocytes and said monocyte selective binding agent; and
(b) removing said complexes from said blood leukocyte composition.

17. The method of claim 16, further comprising contacting granulocytes with a granulocyte selective binding agent under conditions where complexes are formed between said granulocytes and said granulocyte selective binding agent, and removing said complexes from said blood leukocyte composition.

18. The method of claim 1, wherein said depleting consists of:
(a) contacting said B cells with at least one B cell selective binding agent, said T cells with at least one T cell selective binding agent, and said monocytes with at least one monocyte selective binding agent under conditions where complexes are formed between said B cells and said B cell selective binding agent, said T cells and said T cell selective binding agent, and said monocytes and said monocyte selective binding agent; and
(b) removing said complexes from said blood leukocyte composition.

19. The method of claim 16, wherein said B cell selective binding agent binds to a molecule selected from the group consisting of CD19, CD20, CD21, CD22, CD23, CD24, CD37, CD40, CDw75, CD76 and an Ig chain.

20. The method of claim 19, wherein said B cell selective binding agent binds to CD19 or CD20.

21. The method of claim 16, wherein said T cell selective binding agent binds to a molecule selected from the group consisting of CD2, CD3, CD4, CD5, CD6, CD7, CD8, CD27, CD28, CD32, CD43, and a T cell receptor α or β chain.

22. The method of claim 17, wherein said granulocyte selective binding agent binds to a molecule selected from the group consisting of CD66b, CD15 and CD24.

23. The method of claim 21, wherein said T cell selective binding agent binds to CD2 or CD3.

24. The method of claim 16, wherein said monocyte selective binding agent binds to a molecule selected from the group consisting of CDw12, CD13, CD14, CD15, CDw17, CD31, CD32, CD33, CD64, CD98.

25. The method of claim 24, wherein said monocyte selective binding agent binds to CD14.

26. The method of claim 16, wherein
(a) said B cell selective binding agent binds to CD19 or CD20,
(b) said T cell selective binding agent binds to CD2 or CD3, and
(c) said monocyte selective binding agent binds to CD14.

27. The method of claim 16, wherein said B cell selective binding agent, said T cell selective binding agent or said monocyte selective binding agent is an antibody.

28. The method of claim 27, wherein said B cell selective binding agent, said T cell selective binding agent and said monocyte selective binding agent are antibodies.

29. The method of claim 1, wherein said blood leukocytes are not contacted with a binding agent selective for NK cells.

30. The method of claim 1, wherein said blood leukocytes are not subjected to density gradient centrifugation.

31. The method of claim 1, wherein said depleting of said B cells, T cells and monocytes is performed sequentially.

32. The method of claim 1, wherein depleting of said B cells, T cells and monocytes is performed simultaneously.

33. The method of claim 16, wherein said B cell selective binding agent, said T cell selective binding agent or said monocyte selective binding agent is attached to a solid support.

34. The method of claim 33, wherein said solid support is a paramagnetic beau.

35. The method of claim 16, further comprising contacting said B cell-binding agent complex, said T cell-binding agent complex or said monocyte-binding agent with a secondary binding agent attached to a solid support.

36. The method of claim 35, wherein said secondary binding agent is an antibody.

37. The method of claim 35, wherein said solid support is a paramagnetic bead.

38. The method of claim 1, further comprising depleting the cirDC of CD11c− cirDC.

39. The method of claim 1, further comprising depleting the cirDC of CD11c+ cirDC.

40. A method for producing human circulating dendritic cells (cirDC) for therapeutic use, comprising depleting a human blood leukocyte composition of T cells, monocytes and granulocytes.

41. The method of claim 40, wherein said depleting comprises:
(a) contacting: T cells with at least one T cell selective binding agent; monocytes with at least one monocyte selective binding agent; and granulocytes with at least one granulocyte selective binding agent, under conditions where complexes are formed between said T cells and said T cell selective binding agent, said monocytes and said monocyte selective binding agent, and said granulocytes with said granulocyte selective binding agent; and
(b) removing said complexes from said blood leukocyte composition.

42. A cell composition comprising at least 1×10⁶ cirDC for therapeutic use, produced by the method of claim 1.

43. A cell composition comprising at least 1×10⁹ cirDC for therapeutic use, produced by the method of claim 6.
